(19) United States
(12) Patent Application Publication
(10) Pub. No.: US 2008/0261931 A1
(43) Pub. Date: Oct. 23, 2008

(54) Method and Means of Preventing and Treating Sleep Disordered Breathing

(75) Inventors: Jan Hedner, Goteborg (SE); Ludger Grote, Goteborg (SE); Kaj Stenlof, Torslanda (SE)

Correspondence Address: DICKSTEIN SHAPIRO LLP, 1177 AVENUE OF THE AMERICAS (6TH AVENUE), NEW YORK, NY, 10036-2714, US

(73) Assignee: Cereu-Science AB, Goteborg (SE)

(21) Appl. No.: 12/066,994

(22) PCT Filed: Sep. 4, 2006
(86) PCT No.: PCT/SE2006/001010

§ 371 Date: May 30, 2008

(30) Foreign Application Priority Data
Sep. 16, 2005 (SE) 0502044-1

(51) Int. Cl. A61K 31/185; A61K 31/357; A61K 31/42; A61K 31/454; A61K 31/433; A61K 31/522; A61K 31/57; A61P 11/00

(52) U.S. Cl. 514/171; 514/629; 514/455; 514/397; 514/326; 514/363; 514/263.31

(57) ABSTRACT

A method of treating or preventing snoring, obstructive sleep apnea (OSA) and/or central sleep apnea (CSA) comprises administering a pharmacologically effective amount a salt of acetylhomotaurine (AcHT) such as calcium acamprosate (CA) to a patient. Also disclosed is the use of AcHT and CA for the manufacture of a medicament for treating or preventing snoring, OSA and/or CSA and of a diagnostic device, kit or composition; a protective patch comprising AcHT or CA; and a pharmaceutical composition comprising AcHT or CA and an agent capable of alleviating the effects of snoring OSA and/or CSA, in combined amounts effective in the treatment of snoring OSA and/or CSA, and a carrier.

### FIELD OF THE INVENTION

The present invention relates to a method of preventing and treating sleep disordered breathing and to a means for carrying out the method. The present invention also relates to a method of diagnosing sleep disordered breathing and a corresponding means.

### BACKGROUND OF THE INVENTION

Sleep apnea, that is, the temporary cessation of breathing during sleep may be of central, obstructive, and mixed origin, and is often so classified.

Central sleep apnea (CSA) is characterized by complete cessation of the neural activity controlling respiratory musculature.

Obstructive sleep apnea (OSA) is generally defined as an intermittent cessation of airflow at the nose and mouth during sleep. Continuous periods of apnea are termed apneic events. Their duration may vary but, by convention, apneic events of at least 10 seconds in duration are considered significant. However, apneic events may extend up to 2-3 minutes and may cause complete (apnea) or partial (hypopnea) cessation of airflow. In this application the term apnea comprises hypopnea, and the term apneic event comprises hypopneic event. Apneic events, including hypapneic events, may also occur in aggregated form or appear in a complex with a general reduction of ventilation thereby generating continuous or sustained hypoventilation. In the context of the present invention OSA excludes obstruction by foreign objects or by material excreted by the body, such as mucus. In OSA airflow is interrupted despite continuing respiratory neural drive, although the neural control of upper airway muscles appears to be inadequate. OSA occurs as a result of occlusion of the upper airway, usually at the level of the oropharynx, and is the most prevalent form of sleep apnea. Snoring is a prominent symptom of all forms of sleep disordered breathing. Habitual snoring as such may justify treatment.

Mixed sleep apnea consists of an initial central component followed by obstructive apnea. OSA spans over a wide range of upper airway flow changes with the common denominator of one or more of the following; arousal (brief awakening from sleep), alteration of tissue blood gas and pH content as well as endocrine, paracrine, hemodynamic and vascular changes. In its simplest form the condition may be characterized by subtle airflow restriction typically associated with sleep fragmentation resulting in daytime sleepiness or various degree of cognitive dysfunction as well as various symptoms suggestive on non-restorative sleep. OSA associated with daytime symptoms, specifically daytime hypersomnolence, is generally referred to as the Obstructive Sleep Apnea Syndrome (OSAS). Besides hypersomnolence, cognitive and mood changes appear to provide a substantial burden on general health in this condition. Hypersomnolence has been associated with complications including reduced working and driving performance. Moreover, cardiovascular complications, in particular hypertension, cardiac failure, myocardial infarction and stroke are common in OSA. A considerable number, but not all observed patients with OSA are overweight and OSA has been described to frequently coexist with the metabolic syndrome. OSA has been associated with increased insulin resistance, diabetes, obesity, changes in lipid metabolism and increased platelet aggregability. Such symptoms and complications are not confined to severe cases but also observed in cases of partial OSA and in OSA patients without apparent signs of daytime hypersomnolence.

The prevalence of OSA in the adult population depends on clinical laboratory cut-off values applied for the condition. Epidemiological studies suggest that OSA defined as an apnea-hypopnea index (number of apneas per hour of sleep) equal to or higher than 5 occurs in 24% of working adult men and in 9% of adult women. The prevalence of OSA in combination with pronounced daytime symptoms (OSAS) was observed at a rate of 4% in men and 2% in women. The prevalence of minor daytime symptoms induced by discrete sleep-related breathing disturbances is unknown. However, habitual snoring is a common phenomenon reported by 15-25% of the adult population. The full and detailed pathophysiology of OSA is yet to be fully clarified.

Alcoholics appear to be at increased risk of developing sleep apnea. In addition, moderate to high doses of alcohol consumed can lead to narrowing of the air passage during a following sleep period, causing episodes of apnea even in persons who do not otherwise exhibit symptoms of OSA. The general depressant effects on breathing control mechanisms of alcohol may increase the duration of periods of apnea.

The principal forms of treating or preventing OSA include weight loss, surgery of the upper airway, intra-oral mandibular advancement devices and long-term treatment with positive airway pressure (PAP). PAP treatment operates by the generation of a mechanical airway splint counteracting airway collapse. Although technically effective this method is hampered by poor long-term compliance due to poor tolerance and frequent side effects from airway mucous membranes. Surgery and intra-oral mandibular advancement devices are not uniformly effective. In particular surgery has been associated with a considerable relapse of symptoms also in cases with initially excellent treatment results. Various forms of pharmacological treatment, e.g. acetazolamide and other carbonic anhydrase inhibitory agents, tricyclic antidepressants, theophylline, progesterone, agents influencing serotoninergic neurotransmission, cholinesterase inhibitors, zonisamide and topiramate have been employed but have not yet gained wide clinical use.

Thus there is a need for an improved method for treating or preventing OSA and CSA, and also apnea of mixed origin. In particular their pharmacological treatment would offer a definite advantage over the invasive or non-invasive methods used at present, many of which only provide insufficient relief and some of which are cumbersome to the patient.

Calcium acamprosate is disclosed in U.S. Pat. No. 4,355,043, It has been proposed for treating tardive dyskinesia and other movement disorders in U.S. Pat. No.5,952,389. Calcium acamprosate enhances the production of GABA in the central nervous system (CNS). The exact mode of action of acamprosate is incompletely known but the compound binds specifically to the NMDA-receptor complex and modulates CNS glutamate activity. Acamprosate and has also been proposed for the treatment of addiction to drugs of abuse and modification of behaviour associated with addiction to drugs of abuse in U.S. Pat. No. 6,890,951. Calcium acamprosate is useful in the treatment of alcohol dependence (U.S. Pat. No. 6,323,239).

### OBJECTS OF THE INVENTION

It is an object of the present invention is to provide a method of treating snoring, sleep apnea, and other forms of sleep disordered breathing that reduces and/or eliminates at least some of the drawbacks of the methods known to the art.

Another object of the present invention is to provide a means for carrying out the method of the invention.

A further object of the present invention is to provide a method of detecting the presence of OSA in a patient and a means for use in the method.

Further objects of the invention will become apparent from the following short description of the invention, a number of preferred embodiments thereof, and the appended claims.

### SUMMARY OF THE INVENTION

According to the present invention is provided a method of treating or preventing OSA, CSA and/or apnea of mixed origin, comprising the administration of a pharmacologically effective amount of a pharmaceutically acceptable salt of acetylhomotaurine (3-acetamido-1-propanesulfonic acid), in particular the calcium salt (calcium acamprosate) to a patient in need thereof, with the proviso that sleep disordered breathing caused by external mechanical obstruction of the airways, such as by mucus, is excluded. In addition to the calcium salt pharmaceutically acceptable salts of acetylhomotaurine include the sodium salt, the potassium salt, and the magnesium salt.

Acetylhomotaurine or a salt thereof, in particular calcium acamprosate, has not been considered earlier for the treatment of snoring, OSA, CSA or apnea of mixed origin.

A pharmacologically effective amount of a pharmaceutically acceptable salt of acetylhomotaurine, in particular calcium acamprosate, is one that eliminates or substantially reduces the manifestations of snoring, OSA, CSA or apnea of mixed origin during a substantial portion of a single period of sleep of from 15 minutes to 12 hours, such as a during at least 20% or even 80% or more of said period.

The salt of acetylhomotaurine of the invention, in particular calcium acamprosate, can be administered by various routes. The most preferred route is by peroral administration. For this purpose a pharmacologically effective amount of the salt is incorporated into a tablet, a lozenge, a capsule or similar dosage form comprising a pharmaceutically acceptable carrier. Particularly preferred are peroral preparations designed for uptake through the oral mucosa, such as sublingual preparations. Also preferred is trans-dermal administration. Compositions for trans-dermal delivery of calcium acamprosate are disclosed in, for instance, U.S. 2004/0192683 A1, which is incorporated herein by reference, including the literature cited therein. The transdermal formulation is particularly advantageous in regard of simplicity and from a patient comfort standpoint. It may, for instance, take the form of a transdermal patch.

A peroral composition of calcium acamprosate, is marked in, for instance, Sweden, under the trademark Campral®. For preparing further preparations for per-oral administration reference is made to Pharmaceutical Dosage Forms: Tablets. Vol. 1-3, H A Lieberman et al., Eds. Marcel Dekker, New York and Basel, 1989-1990, which hereby is incorporated into this application by reference. In particular specific reference is made to chapter 7 (Special Tablets, by J W Conine and M J Pikal), chapter 8 (Chewable Tablets, by R W Mendes, O A Anaebonam and J B Daruwala), and chapter 9 (Medicated Lozenges; by D Peters).

The pharmacologically effective amount of the pharmaceutically acceptable salt of acetylhomotaurine of the invention, in particular of calcium acamprosate, in oral administration for treatment of sleep disordered breathing will vary depending on factors such as the particular pharmaceutical composition comprising the salt used, the route of administration, the release profile of the pharmaceutical composition, the severity of the disease, individual pharmacokinetic and -dynamic properties as well as the status of the patient. For instance, the dose range for peroral administration of the pharmaceutically acceptable salt of acetylhomotaurine of the invention, in particular of calcium acamprosate, to an adult, otherwise healthy person will be from 500 to 3000 mg per 24 hours; An amount of from 1000 to 2000 mg is envisaged as the normal range used for a peroral administration in OSA. The appropriate dose range can be narrowed by titration in routine experiments. The half-life of acamprosate in plasma is about 15-30 hrs pending dosage, route of administration, and dosing scheme. The half life is extended in subjects with renal impairment.

In addition to the methods of administration of the compound of the invention mentioned above also parenteral, intranasal, and rectal administration can be useful, as well as administration by inhalation.

The timing of the administration of the pharmaceutically acceptable salt of acetylhomotaurine of the invention, in particular of calcium acamprosate, will depend on the formulation and/or route of administration used. In the majority of cases the compound will be administered as a long-term treatment regimen, whereby pharmacokinetic steady state conditions will be reached. Medication for per oral or parenteral administration may also be given in direct relation to a particular sleeping period, for instance from 30 minutes to 2 hours prior to the expected onset of sleep.

According to an advantageous aspect of the invention the pharmaceutically acceptable salt of acetylhomotaurine of the invention, in particular of calcium acamprosate, is combined, in one and the same pharmaceutical preparation, with other compounds that are effective in treatment of snoring, OSA or CSA, exemplified by but not limited to agents used for treatment of overweight or obesity (eg. sibutramine, topiramate, zonisamide, orlistate, rimonabant), acetazolamide and other carbonic anhydrase inhibitory agents, agents influencing serotoninergic neurotransmission, tricyclic antidepressants, theophylline, progesterone and cholinesterase inhibitors, that results in an improved effect due to additive and/or synergistic properties of two or more components of the combination.

According to a preferred aspect of the invention the pharmaceutically acceptable salt of acetylhomotaurine of the invention, in particular calcium acamprosate, is used for diagnosing sleep disorders related to snoring, sleep apnea or other forms of sleep-disordered breathing to dissociate them from other types of sleep disorders. The diagnostic method according to the invention comprises administrating a pharmacologically effective dose of the salt to a person with suspected OSA in increasing amounts prior to or during sleep. The pharmacologically effective dose may be comprised by multiple doses each of which is not pharmacologically effective, so as to provide for titration of pharmacological effect. The observation of a reduction in the severity and/or number of sleep disordered breathing events or reduced sleepiness/increased alertness during daytime following upon such administration is indicative of the presence of obstructive sleep apnea.

The invention will now be explained in more detail by reference to a preferred but not limiting embodiment.

### DESCRIPTION OF PREFERRED EMBODIMENTS

### EXAMPLE 1

### Repeated Dosing Study with Calcium Acamprosate

A study of repeated calcium acamprosate dosing was undertaken in a 52 year old male patient with a BMI of 32.2. This patient had not consumed any alcohol during the 3 months proceeding the current medication period. The patient suffered from moderate OSA (as determined by an AH index in excess of 35 during previous clinical 8-channel overnight monitoring studies). The contribution from central apneas was low and constituted 5% of obstructive events, respectively. A baseline polysomnographic recording (standard sleep montage, nasal pressure recording) was undertaken at baseline and calcium acamprosate therapy was started on 333 mg three times per day orally (Campral® tablets) with an increase to 2 times 333 mg three times per day after two weeks. Key parameters (indices) were recorded at first dosing and after daily dosing for three weeks (Table).

TABLE
Key parameters recorded in an OSA patient treated with a three
times-daily dose of 666 mg calcium acamprosate

| Parameter | | | | | | | |
|---|---|---|---|---|---|---|---|
| (Index) | BMI | AI | HI | AHI | CSAI | MinSat | Sat <90%/hr |
| Week 0 | 32.2 | 18 | 22 | 40 | 3 | 86 | 3 |
| Week 3 | 32.0 | 7 | 9 | 16 | 2 | 91 | 1 |

Abbreviations:
BMI (body mass index, kg/m²),
AI (apnea index, number of obstructive apneic events per hour of sleep),
HI (hypopnea index, number of obstructive hypopneic events per hour of sleep),
AHI (apnea/hypopnea index, number of obstructive apneic/hypopneic events per hour of sleep),
CSAI (central sleep apnea index, number of central apneic events per hour of sleep),
MinSat (lowest oxygen saturation value recorded during the period of sleep),
Sat<90/hr (number of apneic/hypopneic episodes per hour of sleep with an oxygen saturation below 90%).

The number of central and mixed events was low, and seemed to decrease in proportion to the number of obstructive events. Polysomnographically recorded sleep variables were not systematically affected. There was no clinically significant change in total sleep time after administration of calcium acamprosate. The relative proportions of non-REM stage 1+2 and slow wave sleep as well as REM sleep remained unchanged. Already after one week, the patient spontaneously reported a reduction in daytime sleepiness. In addition, the patient reported that there was a notable reduction in snoring that took place early after introduction of the medication. No significant side effects were seen in the study.

These findings demonstrate a potent apnea reducing effect of calcium acamprosate in sleep apnea, an effect that encompass obstructive and seems to encompass central events. Moreover, the beneficial effect on sleep apnea appeared to be maintained over the entire study period. The effect was demonstrably not due to a change in BMI.

### EXAMPLE 2

### Repeated Dosing, Multiple Medication Study with Calcium Acamprosate and a Weight Reducing Agent

At the time of admittance a 63 year old male patient with a previous history of periodic alcohol dependence had a BMI of 33.2 and clinical signs and symptoms indicative of obstructive sleep apnea. A polysomnographic investigation (investigation 1) undertaken at admission showed an AHI of 30. The patient had recently been started on sibutramine (Reductil® capsules, 15 mg once daily) for weight loss. A follow-up sleep investigation (investigation 2) was performed 126 days later when the patient presented a BMI of 31.8. The AHI at this occasion was 19. Calcium acamprosate therapy was started on 333 mg three times per day orally (Campral® tablets) and increased to twice 333 mg three times per day after two weeks. At the end of this treatment period of four weeks (investigation 3) the BMI was marginally changed compared with investigation 2 (31.6) but AHI had decreased to 9.

TABLE
Effects on Body Mass Index and Apnea Hypopnea Index following
a combination of sibutramine for weight reduction and acamprosate

Week

| | −6 | 0 | 6 | 12 | 18 | 22 |
|---|---|---|---|---|---|---|
| Investigation | — | 1 | — | — | 2 | 3 |
| Sibutramine | x | x | x | x | x | x |
| Acamprosate | — | — | — | — | — | x |
| BMI | | 33.2 | | | 31.8 | 31.6 |
| AHI | | 30 | | | 19 | 9 |

Abbreviations:
BMI (body mass index, kg/m²),
AHI (apnea/hypopnea index, number of obstructive apneic/hypopneic events per hour of sleep),
“—” indicates not performed or not given,
“x” indicates on medication.

These findings confirm a potent apnea reducing effect of calcium acamprosate in a patient with sleep apnea. Notably, this therapy may be combined with an agent used for weight reduction and the effects of calcium acamprosate were additive in comparison with that obtained by the weight reducing agent.

1. A method of treating or preventing snoring, obstructive sleep apnea (OSA) or central sleep apnea (CSA) or both, comprising administering a pharmacologically effective amount a pharmaceutically acceptable salt of acetylhomotaurine to a patient in need thereof, with the proviso that treatment or prevention of snoring, sleep apnea, and sleep disordered breathing caused by external mechanical obstruction of the airways is excluded.

2. The method of claim 1, wherein said therapeutically effective dose is such as to be effective during a substantial portion of a single sleep period.

3. The method of claim 2, wherein said substantial portion is 20% or more of said sleep period.

4. The method of claim 2, wherein said substantial portion is 80% or more of said sleep period.

5. The method of claim 2, wherein said single sleep period is from thirty minutes to twelve hours.

6. The method of claim 1, wherein the administration is peroral.

7. The method of claim 6, wherein the administration is sublingual.

8. The method of claim 1, wherein the administration is topical.

9. The method of claim 6, wherein the administration is confined to the frontal portion of the neck and the breast.

10. The method of claim 6, wherein the therapeutically active dose is sustainedly released over a period of time extending from 1 hour to 12 hours.

11. The method of claim 1, wherein from 50% to 100% of said therapeutically effective dose is released within a period of three hours from administration.

12. The method of claim 1, wherein from 80% to 100% of said therapeutically effective dose is released within a period of five hours from administration.

13. The method of claim 10, wherein said therapeutically effective dose is from 100 to 4000 mg.

14.-21. (canceled)

22. A protective patch comprising a pharmaceutically acceptable salt of acetylhomotaurine in an amount therapeutically effective in the treatment of snoring OSA and/or CSA and a pharmaceutically acceptable carrier for transdermal or transmucosal administration.

23. (canceled)

24. A pharmaceutical composition comprising a pharmaceutically acceptable salt of acetylhomotaurine, and a different agent capable of alleviating the effects of snoring OSA and/or CSA, in combined amounts effective in the treatment of snoring OSA and/or CSA, and a pharmaceutically acceptable carrier.

25. The composition of claim 24, wherein said agent is selected from the group consisting of sibutramine, topiramate, zonisamide, orlistate, rimonabant, acetazolamide, an agent influencing serotoninergic neurotransmission, tricyclic antidepressant, theophylline, progesterone and cholinesterase inhibitor.

26. method of claim 1, wherein the pharmaceutically acceptable salt of acetylhomotaurine is calcium acamprosate.

27. The composition according to claim 24 in which the pharmaceutically acceptable salt of acetylhomotaurine is calcium acamprosate.

28. The patch according to claim 22 in which the pharmaceutically acceptable salt of acetylhomotaurine is calcium acamprosate.
